(19) United States
(12) Patent Application Publication
(10) Pub. No.: US 2009/0060993 A1
(43) Pub. Date: Mar. 5, 2009

(54) SOLID PHARMACEUTICAL COMPOSITION FOR ENHANCED DELIVERY OF COENZYME Q-10 AND UBIQUINONES

(76) Inventors: Joseph Schwarz, (US); Michael Weisspapir, (US)

Correspondence Address: ALPHARX INC., 168 KONRAD CRESCENT, SUITE 200, MARKHAM, L3R 9T9, CA

(21) Appl. No.: 11/849,764

(22) Filed: Sep. 4, 2007

(51) Int. Cl. A61K 9/48; A61K 31/122

(52) U.S. Cl. 424/452; 424/94.1

(57) ABSTRACT

The present invention describes a solid oral dosage form of ubiquinones (e.g., ubidecarenone, coenzyme Q-10, idebenone or mixture thereof), providing on contact with water or body fluids the regulated release of an “in situ” formed oil-in-water emulsion with ubiquinone incorporated in the oil phase. Described formulation demonstrates improved bioavailability.

### FIELD OF THE INVENTION

The present invention refers to a process for preparing self-emulsifying with regulated release and enhanced bioavailability tablets comprising coenzyme Q-10 and/or other ubiquinones.

### BACKGROUND OF THE INVENTION

Human mitochondria membranes, as well as other mammalian, contain coenzyme Q homologues with long isoprenoid chains. These coenzymes, under common name of ubiquinones, are the major non-protein components of bioenergetic system of mitochondria, involved in energy-transfer chains, and possess well-known antioxidant potential.

The role of coenzyme Q-10 or ubidecarenone, the most abundant ubiquinone in human body, is to shuttle electrons from complex I or complex II to complex III, affecting directly the oxidative phosphorylation processes for the production of energy (formation of ATP) through metabolic pathways (De Pierre V. C. et al., Ann. Rev. Biochem., 46, 201, 1977; Nakamura T. et al., Chem. Pharm. Bull. 27, 1101, 1979).

Coenzyme Q-10 due to its bioenergetic capacity and antioxidant activity has been widely used in the prophylaxis and therapy of a wide variety of pathological states (Ernster L. et al. Biochim. Biophys. Acta, 1271, 195, 1995; Strijks E. et al., Mol.Aspects Med., 18, S237, 1997).

Idebenone is currently administered to ameliorate cognitive status in patients with clinical history of stroke, Alzheimer's disease, and multi-infarct dementia (Gillis J. C. et al., Drugs Aging, 5, 133, 1994; Gutzman H. et al., J. Neural. Transm. Suppl., 54, 301, 1998). Idebenone has been reported to improve cerebral energy metabolism, to decrease excitotoxic neuronal degeneration, and to stimulate nerve growth factor synthesis (Gillis J. C. et al., Drugs Aging, 5, 133, 1994; Nitta A. et al., Neurosci. Lett., 163, 219, 1993).

Due to its long isoprenoid side chain coenzyme Q-10 and other ubiquinones are extremely lipophilic and insoluble in gastrointestinal fluids. It causes low bioavailability of these compounds when administered in conventional formulations.

Studies in rodents reveal an efficacy of absorption of coenzyme Q-10 from 1 to 8% of the dose (Katayama K., et al., Chem. Pharm. Bull., 250, 2585-2592, 1972; Zhang Y. et al., J. Nutr., 126, 2089-2097, 1996). In humans, the total absorption is likely to be less than 10% (Tomono Y. et al., Intl. J.Clin. Pharmacol. Ther. Toxicol., 24, 536-541, 1986; Lucker, P. W. et al., Biomed. and Clin. Aspects of Coenzyme Q, Ed. By Folkers K. and Yamamura Y. Elsevier Sci.Pub., Amsterdam, pp.143-151, 1984), which is low, but consistent with the data obtained from the animal experiments.

It is well known that lipophilic substances with a very low solubility in water will have a higher bioavailability when administered in a microemulsion (Constantinides, P. P. et al., Journal of controlled release 34, 109-116, 1995). It was shown that the smaller the particle size of the oil droplets, the higher absorption will be achieved (Shah N. H. et al., Intl. J. Pharmaceutics 106, 15-23, 1994).

Emulsions and self-emulsifying drug delivery systems usually comprise a mixture of the liquid or semi-solid lipid phase (usually fatty acid glycerides or esters) with a surfactant (e.g., oxyethylated glycerides or oxyethylated fatty acids), and an additional cosurfactant or cosolvent (e.g., lecithin, monoglycerides, aliphatic alcohols, PEO-PPG copolymers). A hydrophobic drug can be efficiently dissolved in the mixture. After the addition of water, the mixture rapidly converts into an oil-in-water emulsion with the drug remaining in the oil droplets. Absorption of the drug in the gastro-intestinal system from the emulsion is increased. [e.g., Cheng et al., U.S. Patent Application #20030236236]

Microemulsion systems are similar to self-emulsifying systems and often are composed of same components (oil, surfactant, short or medium chain alcohol as the cosolvent), but additionally contain water and have different ratio of the components. When diluted with water, an oil-in-water or water-in-oil emulsion may be produced from microemulsion, accordingly to composition and water amount. For microemulsions drug loading and drug absorption in the stomach and intestine is usually improved. In in-vivo absorption studies in dogs for a lipophilic drug, self-emulsifying delivery system gave at least a 3-fold greater Cmax and AUC (area under curve) than either the drug in solution, a tablet of micronized drug or a capsule of wet-milled spray dried powder (Shah N. H. et al., Intl. J. Pharmaceutics 106, 15-23, 1994).

To improve bioavailability of the lipophilic drugs, and specifically ubiquinones, most of the oral formulations are based on oil and solvent-dissolved drug compositions encapsulated into soft gelatin capsules (Seghizzi, et al., U.S. Pat. No. 5,443,842; Udell et al., U.S. Pat. Nos. 6,616,942; 6,955,820; 7,060,263).

To improve absorption of Coenzyme Q-10 into an intestinal tract, Udell et al. (U.S. Pat. No. 6,616,942) use a formulation comprising Coenzyme Q-10, beta-carotenes, Vitamin E, and medium chain triglycerides in rice bran oil and an optional thickener, such as bee's wax, encapsulated into soft Gelatin Capsules.

Same authors (Udell et al.) suggested slightly different composition comprising Coenzyme Q-10, rice bran oil, a mixture of mono-, di- and tri-glycerides of 16 to 18 carbon chain length with polyglycerol oleate, yellow beeswax, soybean oil, vitamin E and vitamin A. The composition is supplied in a soft gel capsule (U.S. Pat. No. 7,060,263).

Anderson, et al. (U.S. Pat. No. 6,403,116) invented oral composition for animal use comprising coenzyme Q-10 dissolved in methylsulfonylmethane and with addition of citric acid, at least one polysorbate materials, and at least one water-soluble polysaccharide, such as maltodextrin.

All of the delivery systems discussed are liquid preparations and as such, the formulation must be administered as a fluid mixture or as a soft gelatin capsule (SGC). Although useful, liquid and SGC present complications in terms of gelatin safety, compatibility with SGC walls, dosage from stability and manufacturing restraints. The products for oral administration formulated in this way and currently available commercially. They may have high bioavailability but limited stability and consumer compliance.

Tableted forms of emulsions and self-emulsifying drug delivery systems are limited to matrix type tablets, which do not provide any significant improvement of bioavailability. In addition, tablets with a high concentration of oil phase or low melting point lipids and waxes are very soft, demonstrate poor friability and are difficult to manufacture due to sticking, chipping, capping problems and oil leakage during tableting. The described formulations for oil containing tablets correspond to low loaded compositions, with oil levels usually measuring below 20%. (Gupta et al., U.S. Pat. No. 5,591,451; Okada et al, U.S. Pat. No. 5,164,193). Formulations highly loaded with omega-acid rich oil preparations (Desai et al., U.S. Pat. No. 4,867,986) need to be fabricated using a complicated pre-emulsification process, followed by spray-drying and result in a product with poor tablet cohesion.

Use of microcrystalline cellulose, inorganic silicates, silicon dioxide or calcium phosphate as oil sorbents have been described in, for example, U.S. Pat. No. 4,327,076 (Puglia et al) and U.S. Pat. No. 6,562,372 (Yokoi et al.).However, to obtain a free flowing oil-containing composition for tableting, Yokoi used emulsification, followed by spray-drying, without which, tablet formulations could not be prepared.

In U.S. Pat. No. 5,897,876, issued to Rudnic et al., there is disclosed an emulsified drug delivery system which specifically relates to a water-in-oil emulsion which contains a discontinuous water phase in an amount of between 5.1 and 9.9%. The examples are all directed to liquid compositions. Since the compositions are all liquid there is inherently a hydrophilic phase. In terms of tablet or solid discussion, Rudnic et al. only teach that the water emulsion could be absorbed on tablet excipients. This is significantly different from providing a tablet which is a homogenous composition emulsifiable in the presence of body fluid. In this respect, Rudnic et al. disclosure is simply directed to a coating on a preformed tablet. The only area where the composition would be marginally homogeneous would be the exterior layer of the preformed tablet. It is mentioned that formation of the emulsion requires the application of shear force, i.e. this formulation cannot be described as “self-emulsified”.

Tablet manufacturing processes with drugs incorporated into oil-in-water or water-in-oil microemulsions and then absorbed onto solid particles are described in U.S. Pat. No. 6,280,770; (Pather et al., 2001), U.S. Pat. No. 6,692,771 (Pather et al., 2004), and in U.S. Pat. No. 6,379,700 (Joachim et al., 2002). Authors claim improved bioavailability and sustain-release effects. However, the described processes could not provide high drug load per dosage form due to low weight per weight ratio between emulsion and solid carrier material. Increasing the ratio toward liquid emulsion leads to low tablet hardness and high friability.

It is well established that the initial size of the particles constituting a powder is an important factor in determining its compaction behavior. For most powdered materials, compaction of the smaller particles result in stronger tablets because of their larger surface area available for bonding (Sun and Grant, 2001). Common processing excipients, such as colloidal silica and magnesium stearate, are used here as glidant and lubricating agents, respectively. It is well known that magnesium stearate mixing time has a profound effect on dissolution and drug release rate from conventional tablet dosage forms. Among other processing parameters, compaction force was also shown to have an effect on the dissolution rate, hardness and friability of different products (Dabbagh et al., 1996; Hariharan et al., 1997).

Controlled release effect of a self-emulsifying formulation of Coenzyme Q-10 from a tablet dosage form has been confirmed by Nazzal S. et al, 2006. The eutectic-based self-nanoemulsified drug delivery system (SNEDDS) of CoQ10 was prepared as follows. CoQ10 and lemon oil at a ratio of 1:1 were mixed and melted at 37° C. Cremophor EL and Capmul™ MCM-C8 were added to the oily mix at a final concentration of 26.9% (w/w) each. The resultant emulsion was cooled and a viscous paste was mixed with copolyvidone (Kollidon VA 64), then maltodextrin (Glucidex™ IT 12) was added, and the mixture was blended to obtain uniformly sized granules. The granules were then mixed with microcrystalline cellulose (Avicel® PH-112). Then silicon dioxide, used as a glidant, was blended with the mixture. Before compression magnesium stearate was added to the granulation and mixed. A second layer comprising microcrystalline cellulose and magnesium stearate was added and compressed to make a double layered tablet dosage form, in order to increase the hardness of the compacts without modifying the dissolution profile of the tablets. The authors postulated that a tablet dosage form could be manufactured to release a lipid formulation in a controlled release pattern without the need for complicated manufacturing techniques. However, they admitted that elevated temperature and humidity have a devastating effect of physical stability of the preparation. Same process is described in United States Patent Application #20030147927. Process is suitable only for small scale manufacturing (few grams), and maximal Coenzyme Q-10 loading of the tablet is approximately 2% (30 mg per 1245 mg tablet), and tablets have low hardness (<6 kp).

U.S. Pat. No. 7,026,361 has been granted to Minemura, et al. It describes composition comprising ubiquinone and having superior dispersion-stability in an aqueous solution and high bioavailability, as claimed by the inventors. The ubiquinone is dispersed (not dissolved) and emulsified in an aqueous solution of a water-soluble material in the presence of an organic acid(s) to form a protective colloid, the average particle size of the suspended particles being not more than 5 micron. The liquid composition can be adsorbed in an excipient, or dried as is. The inventors prove low particle size of ubiquinone/water colloid particles, high stability of ubiquinone and increased industrial applicability of the proposed invention. However, the bioavailability of the invented composition is questionable, as ubiquinone remains in a poorly soluble form, even with increased surface area.

It is evident that the solid dosage forms, comprising ubiquinone, preferably tablet form of Coenzyme Q-10, with enhanced bioavailability and controlled-release properties and suitable for industrial manufacturing using standard equipment and high-speed tablet presses, to produce tablets with satisfactory hardness and friability, are of high demand.

### SUMMARY OF THE INVENTION

One object of the present invention is to provide a novel composition for solid tablet comprising ubiquinone/s and method of forming this tablet to enhance the bioavailability of an active ingredient over a prolonged period of time.

It has been found that the composition based on proper mixture of ubiquinone/s with oil phase and surfactant (or combination of surfactants) and physiologically acceptable excipients, explicitly specific sorbents, can be successfully fabricated as dry solid tablet. Such tablets can be easily manufactured using standard equipment—mixers, granulators, tablet presses. Being placed into the water-containing media, e.g. gastro-intestinal fluids, the abovementioned tablet generates “in situ” formation of oil-in-water emulsion with the particle size of oil droplets from 10 nm to 10 mcm, and with ubiquinone/s dissolved in these droplets.

The further object of the present invention is to provide lipid phase suitable to obtain high solubility of ubiquinone/s and comprises at least one component selected from the group of edible oils of animal or plant origin, mono- di- and triglycerides, lipid soluble vitamins, and other suitable oily solvents for hydrophobic ubiquinone/s.

The further object of the present invention is to provide at least one surfactant from the group of suitable surfactants in order to obtain the minimal size of the oil droplets once the oil-in-water emulsion will be formed following contact with aqueous media.

It is a further object of the present invention to provide an appropriate sorbent or system of sorbents which allows to absorb relatively high amount of lipid phase and avoid its leaking during the compression, allowing to obtain a freely flowable granulation. It was found the combination of water soluble or water swellable filler and rigid highly porous inorganic sorbent or sorbents resulted in free flow granulation, and yielded hard tablets with no oil leakage and high compressibility.

The further object of one embodiment of the present invention is to provide a solid composition for improved bioavailability of orally delivered ubiquinone/s, said composition being self-emulsifying for forming an oil-in-water emulsion with pre-determined release rate of active ingredient/s. Since such tiny oil droplets mimic chylomicrons, they efficiently absorb in gastro-intestinal tract thus increasing bioavailability of poorly soluble ubiquinones, incorporated in lipid phase.

The pre-determined release rate of ubiquinone/s from tablet could be achieved by introducing water-swellable eroding polymers into tablet composition. These polymers when contacted with gastro-intestinal fluids form a hydrated gel matrix which retains incorporated lipid droplets loaded with ubiquinone/s. The process of gel aqueous dissolution and lipid droplets diffusion into surrounded media both affect release rate of active ingredient. The proper amount of water-swellable eroding polymers helps to achieve a desirable rate of drug release. Besides, the immediate release tablets can also be prepared by using the appropriate disintegrants.

### BRIEF DESCRIPTION OF THE DRAWINGS

FIG. 1 is a graphical representation of the dissolution rate of coenzyme Q-10 self emulsifying controlled release tablet;

FIG. 2 is a graphical representation of the dissolution rate similar to FIG. 1 using a 50 mg tablet;

FIG. 3 is a graphical representation of the dissolution rate of coenzyme Q-10 for different pH;

FIG. 4 is a graphical representation of the dissolution data for a variety of formulations;

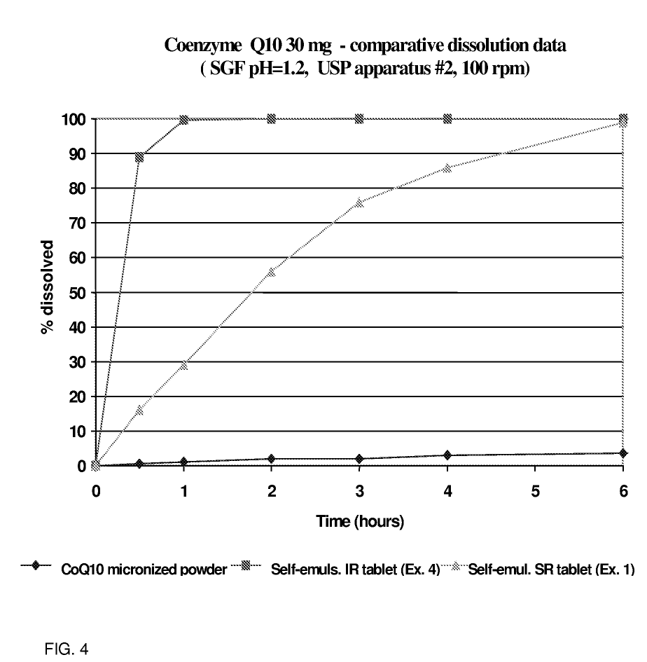

FIG. 5 is a graphical representation of the particle size distribution for a self emulsifying tablet;

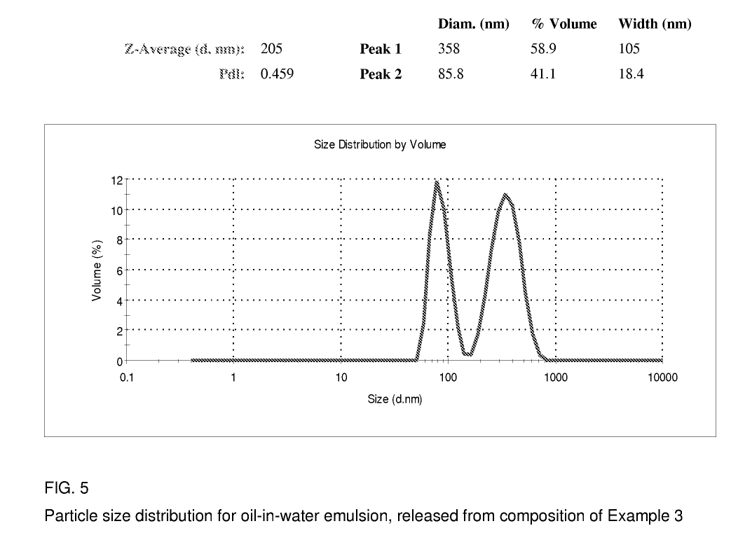

FIG. 6 is a graphical representation of comparative pharmacokinetics for coenzyme Q-10 tablets in healthy volunteers;

FIG. 7 represents comparative dissolution of two 50 mg Coenzyme Q-10 tablets

### DETAILED DESCRIPTION OF THE PREFERRED EMBODIMENTS

The preparation of pharmaceutical composition for manufacturing of tablet dosage form with enhanced bioavailability of coenzyme Q-10 and other ubiquinones includes initial dissolution of the active substance in the lipid phase with addition of surfactant/s.

The lipid phase can be prepared from any physiologically acceptable oily or fatty component(s). It is desirable that the lipid phase is liquid or semisolid at body temperature to form an oil-in-water emulsion. As example, the lipid phase may comprise: triglycerides (food grade oils: olive, corn, canola, soy; palm oil, cocoa butter, fractionated palm oil, medium chain triglycerides (MCT, capric/caprylic glycerides), etc.; animal fats, fish oil, tallow oil, modified glycerides—acetylated monoglycerides, mono- and digylcerides; lipid soluble vitamins—alpha-, beta- and gamma-tocopherol and correspondent tocopherol esters (vitamin E), tocotrienols and related compounds, retinol and retinol esters (vitamin A), etc.; aliphatic and aromatic esters: tributylcitrate, diethyladipate, dibutylphtalate, or miscellaneous lipid substances, such as squalan, squalen, mineral oil, liquid silicon polymers, synthetic and natural waxes with a suitable melting point.

The surfactant/s may be selected from polyethoxylated derivatives of tocopherol acid succinate (TPGS™, Eastman), glycerides (Gellucire™, Gatefosse, Tagat™, Henkel; etc), polyol esters (Sorbitan esters, Tween™), sucrose stearates (Crodesta™, Croda); PEG derivatives of long chain acids (PEG stearate, Lipo-PEG™, Mirj® 52) or block-copolymers (Poloxamer™, Pluronic™) with suitable HLB value.

To form a tablet with suitable physico-chemical properties, an appropriate sorbent for the lipid phase must be used. The sorbent function is to hold the lipid phase during the granulation process to provide free flowing granulation and prevent the lipid phase from leaking during the tableting process. The sorbent should be physiologically inert, safe and suitable for granulation and tableting processes. The sorbent should possess high surface area/porosity, high mechanical strength and be relatively inert to prevent chemical interaction with formulation components. As example, the following compounds are typically suitable:

Silicon dioxide—colloidal (dried silicagel—Syloid™ 244, GRACE; Sipernats®, DEGUSSA) or fumed (prepared by hydrolysis of silicone halides -Cab-O-Sil® M5, CABOT, or Aerosil® 200/300, DEGUSSA), inorganic sorbents such as synthetic Magnesium Aluminum Silicate (Neusilin®, FUJI), di- and tribasic calcium phosphates, calcium carbonate, calcium silicate, zeolites, talcite, kaolin, benthonite, etc., cross-linked polymers with high surface area, such as cross-linked povidone (Povidone® XL, BASF) may also be used.

In respect of suitable excipients, sorbents, tablet forming materials, glidants, lubricants, hydration regulators can be selected according to desired tablet properties and loading level. Since many of the proposed components are liquid or semisolid materials at room temperature, preparation of the tablets becomes a challenging task. Highly absorptive compounds facilitate for preparation of free flowing powders, however, most of the absorbed material is squeezed out of the matrix during tablet compression (applied force is typically 1-10 tons per tablet), thus compromising the properties of the tablet.

It was found that the combination of microcrystalline cellulose (polysaccharide type sorbent) with inorganic sorbents, resulted in a preparation with good flowability, without water granulation, avoiding oil leakage during tableting, and provides tablets with high hardness and excellent friability. The selection of the ratio between the microcrystalline cellulose, lipid phase and inorganic sorbents results in tablets with desired properties.

The current invention describes the preparation of tablets with a high lipid and surfactant content. The tablets possess acceptable physical characteristics such as hardness, friability, dissolution behaviour and can be manufactured using standard equipment such as granulators, ovens, dryers, mixers, tablet presses. On contact with water media, the tablets release “in-situ” forming oil-in-water emulsions comprising active components dissolved in the oil phase.

Such properties facilitate high bioavailability for hydrophobic substances included into the tablet.

Polymers for release rate control work as main dissolution rate regulators. After contact with water they form a hydrated gel in parallel with emulsification process. Release of the formed emulsion follows the gel dissolution and partial diffusion of the tiny lipid droplets from gelled matrix to surrounded media. Preferred gel forming polymers are water swellable or water soluble cellulose derivatives, for example,

Hydroxypropylmethylcellulose (Methocel™, types A, E, K, F, Dow Chemical), Hydroxyethylcellulose (Natrosol™, Hercules), Hydroxypropylcellulose (Klucel™, Aqualon), Carboxymethylcellulose (cellulose gum). Another types of synthetic polymers include polyacrylic acid (Carbopol™, BFGoodrich), Polyethylene oxide (Polyox™, Union Carbide), Polyvinylpyrrolidone (Kollidon™ and Povidone™ PVP and PVP-VA, ISP and BASF), natural gums and polysaccharides—Xantan gum (Keltrol™, Kelco), carrageenan, locust bean gum, acacia gum, chitosan, alginic acid, hyaluronic acid, pectin, etc.

### EXAMPLES

### Example 1

### CoQ10 Self-Emulsifying Controlled Release Tablet; 30 ml Strength, Dissolution Time Greater Than 6 Hours

As a first example of the first formulation, the slowly dissolving composition contains CoQ10 (Ubiquinone) in amount of 30 mg per tablet. The oil phase comprises of alpha-tocopherol acetate (vitamin E acetate), PEG-40 stearate (Lipo-PEG 39S) used as the surfactant with optimal HLB value for effective emulsification of the oil phase. A weight ratio of 1:1 between CoQ10 and the oil phase was used. In respect of the surfactant to oil phase, the w/w ratio used was 1.6 to 1.

The composition of the 30 mg CoQ10 self-emulsifying extended release tablet is displayed in table 1.

TABLE 1
Pharmaceutical Solid Self-Emulsifying Composition for Sustained
Delivery of Coenzyme Q-10 (30 mg tablet)

| INGREDIENTS | Per tablet, mg | % |
|---|---|---|
| Coenzyme Q-10 | 30 | 6.41% |
| Tocopherol acetate | 30 | 6.41% |
| PEG-40 stearate | 50 | 10.68% |
| Dibasic calcium phosphate | 15 | 3.21% |
| Colloidal silicon dioxide (Cab-O-Sil ®) | 45 | 9.62% |
| Lactose (spray dried) | 110 | 23.50% |
| Methocel ™ E-15 | 24 | 5.13% |
| (Hydroxypropylmethylcellulose) | | |
| Methocel ™ K4M | 48 | 10.26% |
| (Hydroxypropylmethylcellulose) | | |
| Microcrystalline cellulose (Vivapur ™ 102) | 90 | 19.34% |
| PEG 8000 | 18 | 3.85% |
| Povidone (PVP K-25) | 6 | 1.28% |
| Magnesium stearate | 2 | 0.43% |
| Tablet weight: | 468 | 100.0% |

### Preparation

CoQ10, surfactant (PEG stearate) and oil phase (alpha-tocopherol acetate) were heated together between 50° C. and 55° C. and mixed until the coenzyme completely dissolved. This solution was diluted with ethyl alcohol and then mixed with colloidal silicon dioxide, dibasic calcium phosphate and part of microcrystalline cellulose as sorbents. The paste was carefully mixed to obtain homogenous dispersion. This is important to maintain a relatively uniform composition in the final tablet and also contributes to prolonged release and bioavailability. This dispersion was transferred to a planetary granulator and carefully mixed with gel-forming.

Hydroxypropylmethylcellulose polymers Methocel™ K4M, Methocel™ E15 and part of lactose (hydration rate regulator). The mixture was granulated with separately prepared 5% binder solution of polyvinylpyrrolidone (Povidone™ PVP K-25) in ethyl alcohol until a suitable granulate was obtained. This granulation was dried at 45° C. until the solvent evaporated. The dry granulation was passed through a 16 mesh sieve, mixed with microcrystalline cellulose, lactose and sieved magnesium stearate (lubricant).

Tablets were prepared using conventional equipment (such as 16-station rotary tablet press). The tablets had a hardness greater than 8 kg and friability of less than 1%.

Dissolution tests were carried according to USP requirements, using USP apparatus #2 at 37° C., with paddle rotation at 100 rpm. 900 ml of simulated gastric fluid (SGF) without enzymes or simulated intestinal fluid (SIF) served as the dissolution media.

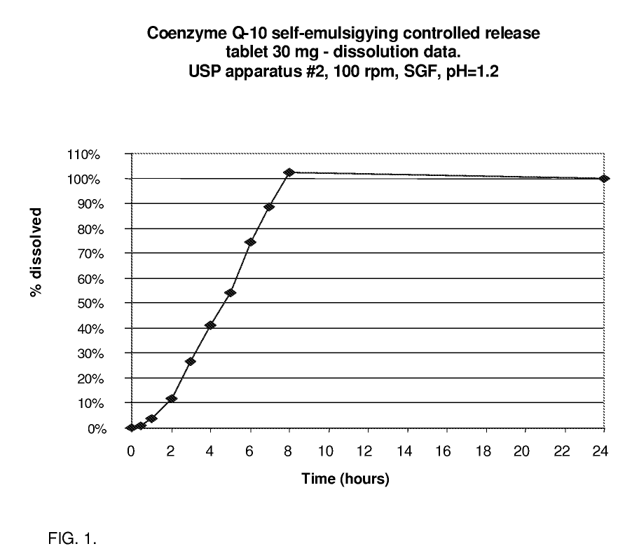

Dissolution was insensitive to media type. The tablet was almost completely dissolved between 6 and 8 hours. Upon dissolution, a colloidal emulsion of the CoQ10 dissolved in the oil phase was formed and gradually released into dissolution media, forming a hazy bluish dispersion. The dissolution pattern is displayed in FIG. 1.

### Example 2

### CoQ10 Self-Emulsifying Controlled Release Tablet (50 mg Strength)

TABLE 2
Tablet Composition Pharmaceutical Solid Self-Emulsifying
Composition for Sustained Delivery of CoQ10 (50 mg tablet)

| INGREDIENTS | Per tablet, mg | % |
|---|---|---|
| Coenzyme Q-10 crystalline | 50 | 5.75% |
| alpha-Tocopherol acetate (Vitamin E acetate) | 50 | 5.75% |
| PEG-40 stearate | 50 | 5.75% |
| Tocophersolan USP | 30 | 3.45% |
| Neusilin US2 (Fuji Chemicals) | 85 | 9.77% |
| Dibasic calcium phosphate anhydrous | 60 | 6.90% |
| Microcrystalline cellulose (Vivapur ™ 102) | 100 | 11.49% |
| Methocel ™ E-15 | 100 | 11.49% |
| (Hydroxypropylmethylcellulose) | | |
| Methocel ™ K4M CR grade | 50 | 5.75% |
| Mannitol | 250 | 28.74% |
| Povidone (PVP K-25) | 20 | 2.30% |
| PEG-8000 | 20 | 2.30% |
| Magnesium stearate | 5 | 0.57% |
| Tablet weight | 870 | 100.0% |

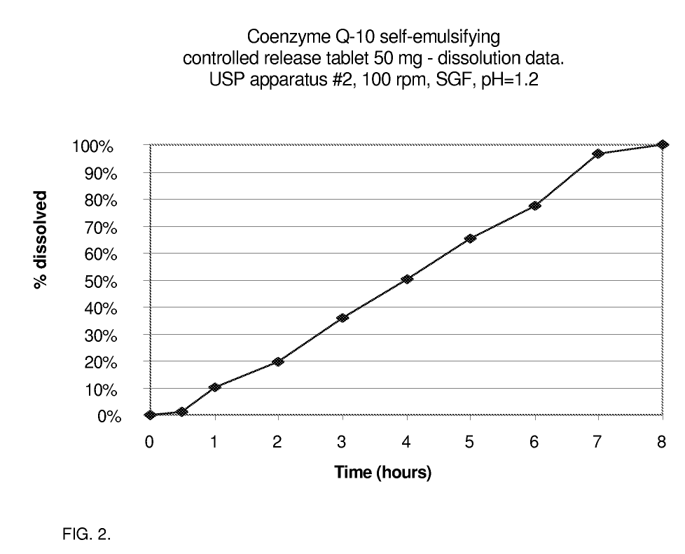

Preparation followed the protocol as described in Example 1. The tablet hardness was found to be between 6 kg and 10 kg with a friability of less than 1%. The dissolution pattern is presented in FIG. 2.

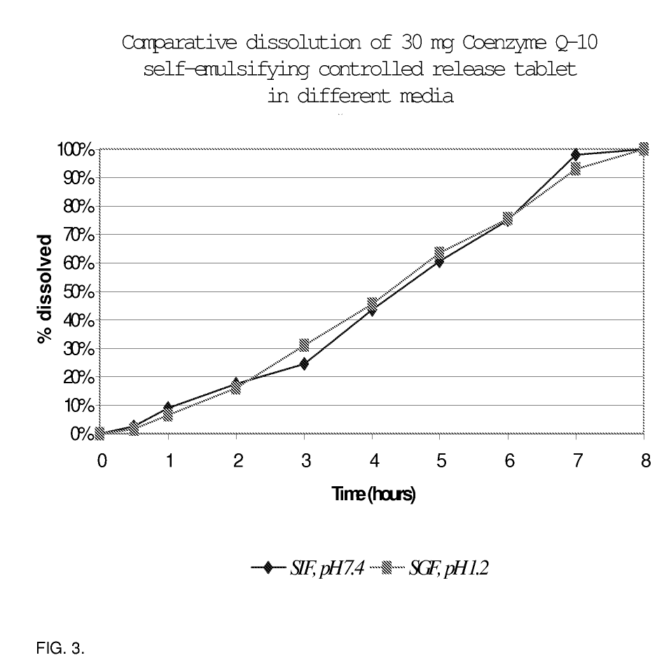

The drug release from self-emulsifying matrix is practically independent to media type. FIG. 3 represents the dissolution pattern in acidic and basic conditions (simulated gastric and intestinal fluids without enzymes, according to USP).

### Example 3

### Idebenone Self-Emulsifying Chewable Tablet (50 mg Strength)

TABLE 3
Tablet Composition Pharmaceutical Solid Self-Emulsifying
Composition for Idebenone chewable tablet

| INGREDIENTS | Per tablet, mg | % |
|---|---|---|
| Idebenone | 50 | 4.00% |
| alpha-Tocopherol acetate (Vitamin E | 50 | 4.00% |
| acetate) | | |
| PEG-40 stearate | 30 | 2.40% |
| TPGS (Tocopherol PEG succinate) | 20 | 1.60% |
| Ethyl alcohol (for granulation only) | q.s. | N/A |
| Maltodextrin | 120 | 9.60% |
| (Silicon dioxide) Sipernat ™ DEGUSSA | 100 | 8.00% |
| Dibasic Calcium phosphate anhydrous | 150 | 12.00% |
| Microcrystalline cellulose Vivapur ® 102 | 180 | 14.40% |
| Mannitol + Xylitol mixture 1:1 | 500 | 40.00% |
| Povidone (PVP K-90) | 40 | 3.20% |
| Magnesium stearate | 10 | 0.80% |
| Tablet weight | 1250 | 100.00% |

Chewable Self-emulsifying Idebenone tablet was prepared by granulation of all components with ethyl alcohol in appropriate blender, followed by drying of the formed granulation in oven (55° C.) or using fluid bed drier. After compression tablet has hardness >10 kp and low friability.

### Example 4

### Coenzyme Q-10 Self-Emulsifying Chewable Tablet (50 mg Strength)

TABLE 4
Tablet Composition Pharmaceutical Solid Self-Emulsifying
Composition for Coenzyme Q-10 immediate release tablet

| INGREDIENTS | Per tablet, mg | % |
|---|---|---|
| Coenzyme Q-10 crystalline | 50 | 6.96% |
| alpha-Tocopherol acetate (Vitamin E acetate) | 50 | 6.96% |
| PEG-40 stearate (Lipo-PEG 39) | 80 | 11.14% |
| Colloidal silicon dioxide (Cab-O-Sil) | 25 | 3.48% |
| Calcium silicate (Huberderm ™ 1000) | 120 | 16.71% |
| Dibasic calcium phosphate | 50 | 6.96% |
| Microcrystalline cellulose (Vivapur ™ 102) | 120 | 16.71% |
| Methocel ™ E-15 LV Premium grade | 40 | 5.57% |
| Lactose (spray dried) | 150 | 20.89% |
| Povidone (PVP K-25) | 10 | 1.39% |
| Alcohol for granulation | q.s. | N/A |
| Crosspovidone (PVP XL-10) | 30 | 4.18% |
| Magnesium stearate | 3 | 0.42% |
| Tablet weight | 718 | 100.0% |

Tablet was prepared using alcohol granulation, as described in Example 3. Tablet with hardness 7-9 kp had disintegration time in range 14-23 minutes (USP method) in water.

### Example 5

### Coenzyme Q-10 Self-Emulsifying Tablet (50 mg Strength)—Melt Granulation

TABLE 5
Pharmaceutical Solid Self-Emulsifying Composition for Coenzyme
Q-10 tablet

| INGREDIENTS | Per tablet, mg | % |
|---|---|---|
| Coenzyme Q-10 crystalline | 50 | 5.95% |
| alpha-Tocopherol acetate (Vitamin E | 50 | 5.95% |
| acetate) | | |
| PEG-40 stearate (Mirj ® 52) | 50 | 5.95% |
| Tocophersolan USP | 30 | 3.57% |
| Neusilin US2 (Fuji Chemicals) | 85 | 10.12% |
| Dibasic calcium phosphate anhydrous | 60 | 7.14% |
| Methocel E-15 LV Premium grade | 100 | 11.90% |
| Methocel K4M CR grade | 50 | 5.95% |
| Mannitol | 300 | 35.71% |
| Povidone (PVP K-90) | 20 | 2.38% |
| Polyethylene oxide 8000 | 40 | 4.76% |
| Magnesium stearate | 5 | 0.60% |
| Tablet weight | 840 | 100.00% |

PEG-40 stearate, Tocophersolan and alpha-Tocopherol acetate were combined and heated together at 55°-60° C. until melted. Crystalline coenzyme Q-10 was added to melted mixture and dissolved completely. Neusilin® US2 was added slowly with continuous mixing to melted mass with stirring until homogeneous mixture is formed. Then dibasic calcium phosphate, Methocel E-15, Methocel K4M, Mannitol, Povidone K-25 and PEG 8000 were added and mixed well. After mixing formed granulation was screened through 12 mesh stainless screen and cooled to room temperature. Magnesium stearate was added and mix well using appropriate blender. To improve tabletting properties, granulation can be compacted or slugged, then milled and compressed into tablets. Tablets (oval or capsule shape tablets, hardness 6-8 kp, target tablet weight 840 mg) were compressed using suitable tablet press machine.

Particle size distribution was measured using Zetasizer particle size analyzer Nano ZS model ZEN3600 (Malvern Instruments Ltd.) Measurement parameters and settings were entered manually according to manufacturer recommendations.

### Graph 5

Particle size distribution for oil-in-water emulsion, released from composition of Example 3

| | | | Diam. | % | Width |
|---|---|---|---|---|---|
| | | | (nm) | Volume | (nm) |
| Z-Average (d. nm): | 205 | Peak 1 | 358 | 58.9 | 105 |
| Pdl: | 0.459 | Peak 2 | 85.8 | 41.1 | 18.4 |

As presented at Graph 5, particle size distribution of the o/w emulsion, released from composition Example 3 mimics size distribution of chylomicrons [Fraser R., “Size and lipid composition of chylomicrons of different Svedberg units of flotation”, J Lipid Res. 1970 No.1 1(1):60-5]

### Advantageousness of the Solid Self-Emulsifying Composition in Tablet Form

Tablet as dosage form is preferable because of easiness of swallowing, safety of used components (absence of gelatin and other substances of animal origin) and convenience of manufacturing using modern high speed tablet presses. Advantage of sustained release delivery of self-emulsifying compositions is in highly increased bioavailability of the included active components. It is very important for poorly soluble compounds. Moreover, controlled delivery of such compounds can significantly decrease potentially dangerous drug dumping and provide constant and uniform input.

Entrapping of the drug into the small oil droplets, which mimic chylomicrons and could be absorbed by similar mechanism, visibly increases penetration efficacy through the gastrointestinal mucosal membranes. The described composition has sufficient loading of the poor water-soluble drug, and providing a prolonged release of the included drug. Ubiquinone loaded oil-in-water emulsion is gradually released from the sustained release composition, providing optimal supply of incorporated component.

Sustained release of the active material allows change multiple dosing (2-6 tablets a day) to single dose delivery per day. It is much more convenient for patient and decreases chances for dose missing or significant variations of blood level of the drug.

Good mechanical and tabletting properties of the formulation allows to use standard high speed pharmaceutical equipment and approved excipients to manufacture tablets.

### Pharmacokinetics Of Coenzyme Q-10 in Self-Emulsifying Tablet

CoQ10 pharmacokinetics for self-emulsifying tablet (Example 2) was investigated in comparison to the only available 50 mg CoQ10 tablet (Enzymatic Therapy®, CoQ10 50 mg, lot L9300). This tablet contains micronized CoQ10. Experiment was carried in Pharmaceutics School of Department of Pharmaceutical Sciences (Peking University, Beijing 100083, China).

Twenty healthy male volunteers (aged 19-23 years) participated, and a written informed consent agreement was made from every subject. Each subject was determined to be in good health and good mental status through medical history, physical examination, electrocardiograph examination, and routine laboratory profiles such as hematology and blood chemistries. Cigarettes, alcohol, and other drugs were not allowed prior to one week and during the period of experiment.

Each subject received every day tablets of 50 mg of coenzyme Q₁₀ as sustained release tablets in one group (n=12) and regular tablets of the same strength in the other group (n=12). Duration of the experiment was 15 days. The blood samples were taken prior to the oral administration and at specified time point. After removing the proteins by methanol precipitation the plasma was extracted with hexane. Aqueous and organic solvents were separated by low speed centrifugation, organic phase was collected, dried under nitrogen stream and dissolved in 100 μl of ethanol. The solution was injected into HPLC-UV system. Reverse phase ODS column (10μm, 250×4.6 mm) temperature was maintained at 30° C. Mobile phase was constituted by methanol-ethanol 9:1 v/v. Flow rate was 1.5 ml/min and UV detection was carried at 275 nm. Coenzyme Q₉ was used as an internal standard.

Results: Total coenzyme Q₁₀ concentrations in plasma following oral administration of self-emulsified tablets were higher (p<0.05), compared to those in plasma following oral administration of regular tablets. According to obtained pharmacokinetic data, blood concentration of CoQ10 at day 14 increases from initial level ˜50% for commercial immediate release tablet and ˜80% for self-emulsifying tablet. AUC values are 146% and 188%, respectively (100%—initial COQ10 level, 0.81 and 0.96 mcg/ml, resp.).

| | | “Enzymatic Therapy” Lot |
|---|---|---|
| | Self-emulsifying tablet | L9300 |
| | 50 mg Coenzyme Q-10 | micronized CoQ10 50 mg |
| Cmax | 1.85 mcg/ml (day 14) | 1.37 mcg/ml (at day 7) |
| Relative AUC | 361 mcg * hr/ml | 193 mcg * hr/ml |

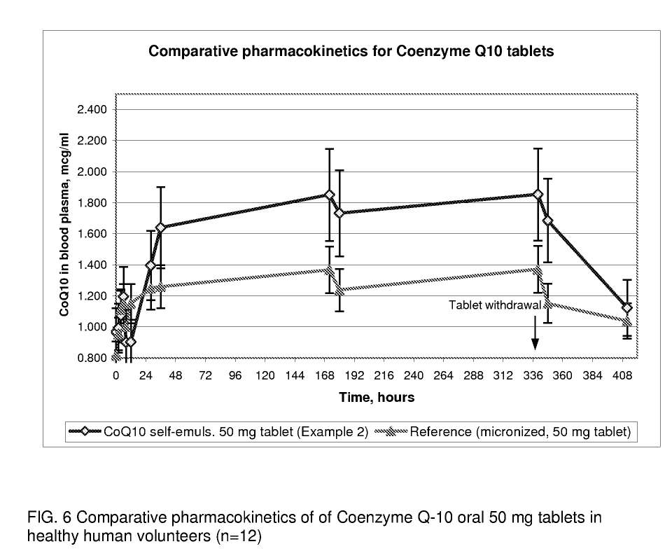

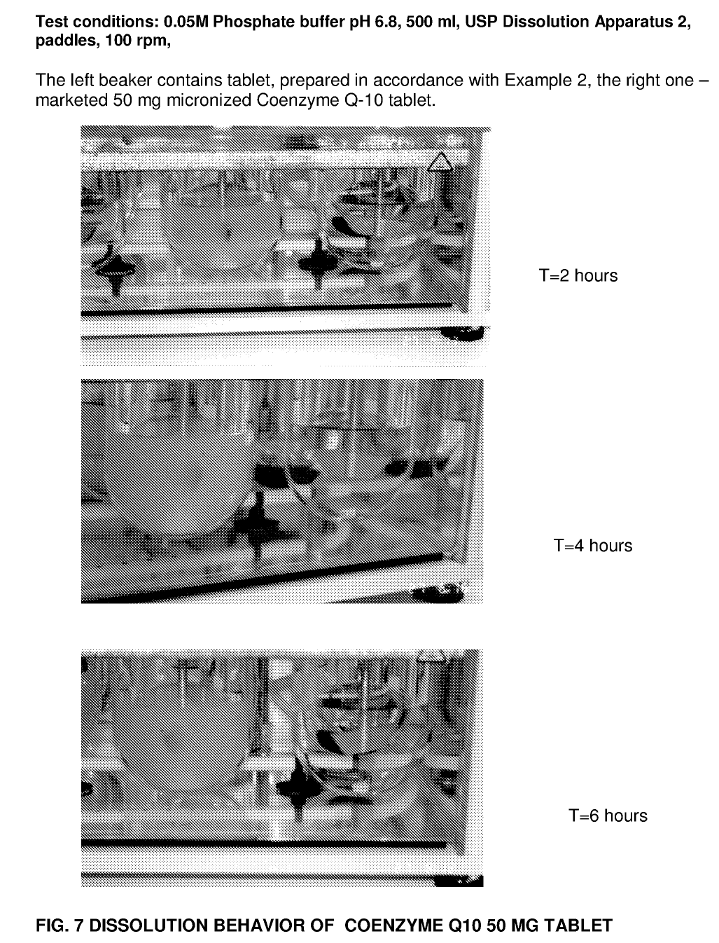

FIG. 6 represents comparative changes in COQ10 concentration in blood plasma after oral administration of two different 50 mg tablets. Dissolution of these tablets is presented at FIG. 7. It is clearly visible the prolonged (sustained) release of emulsion from self-emulsifying tablet and absence of drug release and dissolution from marketed comparator tablet, comprised of micronized Coenzyme Q-10

### REFERENCES

### US Patents

1. Seghizzi, et al., U.S. Pat. No. 5,443,842
2. Udell et al., U.S. Pat. Nos. 6,616,942; 6,955,820; 7,060,263
3. Anderson, et al., U.S. Pat. No. 6,403,116
4. Gupta et al., U.S. Pat. No. 5,591,451;
5. Okada et al, U.S. Pat. No. 5,164,193
6. Desai et al., U.S. Pat. No. 4,867,986
7. Puglia et al., U.S. Pat. No. 4,327,076
8. Yokoi et al., U.S. Pat. No. 6,562,37
9. Rudnic et al., U.S. Pat. No. 5,897,876
10. Pather et al., U.S. Pat. Nos. 6,280,770; 6,692,771
11. Joachim et al., U.S. Pat. No. 6,379,700
12. Khan et al., U.S. Patent Application #20030147927
13. Minemura, et al., U.S. Pat. No. 7,026,361
14. Cheng et al., U.S. Patent Application #20030236236

### Articles

1. DePierre J W, Ernster L., “Enzyme topology of intracellular membranes”, Ann. Rev. Biochem., 1977, No. 46, pp.201-262.
2. Nakamura T, Sanma H, Imabayashi S, Sawa Y Hamamura K. “Effects of exogenous ubiquinone-10 on endogenous ubiquinone-10 in canine plasma and on electron transport enzymes in leucocytes” Chem Pharm Bull (Tokyo). 1979, No. 27(5), pp.1101-5.
3. Ernster, L. and Dallner, G. Biochemical, physiological and medical aspects of ubiquinone function. Biochem. Biophys. Acta, 1271, 195, 1995.
4. Strijks, E., Kremer, H. P., and Horstink, M. W. Q₁₀ therapy in patients with idiopathic Parkinson's disease. Mol. Aspects Med., 18, S237, 1997.
5. Gillis, J. C., Benfield, P., and McTavish, D. Idebenone. A review of its pharmacodynamic and pharmacokinetic properties, and therapeutic use in age-related cognitive disorders. Drugs Aging, 5, 133, 1994.
6. Gutzman, H. and Hadler, D. Sustained efficacy and safety of idebenone in the treatment of Alzheimer's disease: update on a 2-year double-blind multicentre study. J. Neural. Transm. Suppl., 54, 301, 1998.
7. Nitta A., Hasegawa, T., and Nabeshima, T. Oral administration of idebenone, a stimulator of NGF synthesis, recovers reduced NGF content in aged rat brain. Neurosci. Lett., 163, 219, 1993
8. Katayama K., and Fujita, T. Studies on lymphatic absorption of 1′,2′-(³H)-coenzyme Q₁₀ in rats. Chem. Pharm. Bull., 250, 2585-2592, 1972.
9. Zhang Y., Turunen, M., and Appelkvist, E.-L. Restricted uptake of dietary coenzyme Q is in contrast to the unrestricted uptake of a α-tocopherol into rat organs and cells. J. Nutr., 126, 2089-2097, 1996.
10. Tomono Y., Hasegawa, J. and Seki, T., et al. Pharmacokinetic study of deuterium-labelled coenzyme Q₁₀ in man. Intl. J.Clin. Pharmacol. Ther. Toxicol., 24, 536-541, 1986;
11. Lucker, P. W., Wetzelberger, N., and Hennings, G. et al. Pharmacokinetics of coenzyme ubidecarenone in healthy volunteers, in Biomed. and Clin. Aspects of Coenzyme Q, Ed. By Folkers K. and Yamamura Y. Elsevier Sci.Pub., Amsterdam, pp.143-151, 1984.
12. Constantinides, P. P. et al., Journal of controlled release 34, 109-116, 1995.
13. Shah N. H., Carvajal, M. T., Patel, C. I., et al. Self-emulsifying drug delivery systems (SEDDS) with polyglycolyzed glycerides for improving in vitro dissolution and oral absorption of lipophylic drugs. Intl. J. Pharmaceutics 106, 15-23, 1994.
14. Sun C., Grant D. “Influence of Elastic Deformation of Particles on Heckel Analysis” Pharm. Dev. Technol., 2001, vol. 6(2), 193-200
15. Dabbagh M A, Ford J L, Rubinstein M H, Hogan J E. “Effects of polymer particle size, compaction pressure and hydrophilic polymers on drug release” International Journal of Pharmaceutics, 1996, vol. 140 (1), pp. 85 95.
16. Hariharan M, Wheatley T A, Price J C. “Controlled-release tablet matrices from carrageenans: compression and dissolution studies”. Pharm Dev Technol. 1997 (2), pp.383-393.

1. A pharmaceutical composition for manufacturing of solid dosage form with enhanced bioavailability of coenzyme Q-10 and other ubiquinones, wherein said solid dosage form is a tablet with hardness no less than 6 kp

2. Solid dosage form as set forth in claim 1 wherein said tablet dosage form releases oil-in-water emulsion on contact with water media

3. Solid dosage form as set forth in claim 2, wherein particle size of oil droplets in said oil-in-water emulsion is from 10 nm to 10 micrometers

4. Solid dosage form as set forth in claim 1, comprises of:
(i) at least one ubiquinone, selected from the group of coenzymes Q (coenzymes Q3-Q15, preferably coenzyme Q-10) and idebenone, and
(ii) a physiologically acceptable hydrophobic phase, wherein named ubiquinone is dissolved or uniformly dispersed
(iii) at least one physiologically acceptable surfactant
(iv) physiologically acceptable mixture of sorbents to incorporate hydrophobic phase
(v) physiologically acceptable excipients for regulation of dissolution and release rate

5. Solid dosage form as set forth in claim 4, wherein said dosage form is compressed tablet, prepared from granulation

6. A granulation for compressed tablet, as set forth in claim 5, which can be compressed using high speed tabletting machines and said tablet has hardness not less than 6 kp

7. Solid dosage form as set forth in claim 4, wherein said hydrophobic phase comprises at least one component, selected from the group of edible oils of animal or plant origin, mono- di- and triglycerides, acetylated monoglycerides, pharmaceutically acceptable esters of aliphatic hydroxyacids, fatty acids; tocopherol and tocopherol esters, glycol esters, squalane, squalene, limonene, crill oil, oregano oil and lipid soluble vitamins.

8. Solid dosage form as set forth in claim 7, wherein said hydrophobic phase is alpha-D-tocopherol or mixed tocopherols of natural or synthetic origin

9. Solid dosage form as set forth in claim 7, wherein said hydrophobic phase is alpha-D-tocopherol acetate, D,L-tocopherol acetate or mixture thereof

10. A composition for manufacturing of solid dosage form as set forth in claim 4 where ratio between sorbent and hydrophobic phase precludes squeezing of the named hydrophobic phase during tablet compression step

11. Solid dosage form as set forth in claim 4 wherein ratio between sorbent mixture and hydrophobic phase is in range from 1:10 to 10:1, preferably in range 1:3 to 3:1

12. Solid dosage form as set forth in claim 11, where said sorbent mixture comprises of inorganic sorbent and water swellable or water soluble filler

13. Solid dosage form as set forth in claim 12, where said water swellable or water soluble filler is selected from group of polymers, such as microcrystalline cellulose, amorphous cellulose, milled cellulose, hydroxyethylcellulose, hydroxypropylmethylcellulose, hydroxypropyl-cellulose, methylcellulose, carboxymethylcellulose, starch, dextrins, polysaccharides, polyvinyl alcohol or polyvinylpyrrolidone, mono-, di- and polysaccharides and polyols

14. Solid dosage form as set forth in claim 11, where said inorganic sorbent comprises of components, selected from the group of finely dispersed: Silicone Dioxide, Magnesium and Aluminium oxides and hydroxides; inorganic salts, selected from group of Calcium, Magnesium and Aluminium silicates, di-and tribasic Calcium phosphates, Calcium silicates, Talc or mixtures thereof

15. A process for preparation of composition of claim 1 includes distribution of the ubiquinone and surfactant in hydrophobic base, blending of the formed mixture with sorbent(s), following addition of the other excipients, granulation and filling to the capsule or tablet compression

16. A process of claim 15 where said ubiquinone is dissolved or dispersed in melted mixture of hydrophobic phase and surfactants and then mixed with a sorbent

17. A process of claim 15 where granulation is prepared by compacting of sorbent with active components, hydrophobic phase with surfactant(s) and other excipients using compacting or slugging equipment

18. A process of claim 15 where active material is granulated with other components using volatile solvent, followed by subsequent elimination of the solvent.

19. A process of claim 18 wherein said volatile solvent is selected from group of ethyl alcohol, isopropyl alcohol, ethylacetate, acetone, water or mixture thereof

20. Solid dosage form as set forth in claim 5, wherein said compressed tablet is chewable tablet, fast disintegrating tablet, immediate release tablet, sustained release tablet or enterosoluble tablet

20. Solid dosage form as set forth in claim 4, wherein said capsule is hard gelatin capsule, hydroxypropylmethylcellulose capsule, starch capsule, or enterocoated capsule.

21. Solid dosage form as set forth in claim 4, wherein said physiologically acceptable surfactant is selected from group of non-ionic, anionic or amphoteric surfactants
